(19) United States
(12) Patent Application Publication
(10) Pub. No.: US 2014/0308669 A1
(43) Pub. Date: Oct. 16, 2014

(54) METHODS FOR OBTAINING SINGLE CELLS AND APPLICATIONS OF SINGLE CELL OMICS

(71) Applicants: Xing Yang, San Diego, CA (US); David M. Nelson, San Diego, CA (US); Peter Kuhn, Solana Beach, CA (US); Daniel Chesnaye Lazar, San Diego, CA (US)

(72) Inventors: Xing Yang, San Diego, CA (US); David M. Nelson, San Diego, CA (US); Peter Kuhn, Solana Beach, CA (US); Daniel Chesnaye Lazar, San Diego, CA (US)

(73) Assignee: Epic Science, Inc., La Jolla, CA (US)

(21) Appl. No.: 14/001,154

(22) PCT Filed: Jan. 23, 2012
(86) PCT No.: PCT/US12/22248

§ 371 Date: Jun. 12, 2014

Related U.S. Application Data
(60) Provisional application No. 61/435,704, filed on Jan. 24, 2011.
(60) Provisional application No. 61/435,724, filed on Jan. 24, 2011.
(60) Provisional application No. 61/435,721, filed on Jan. 24, 2011.

(51) Int. Cl. C12Q 1/68; G01N 33/574

(52) U.S. Cl. CPC C12Q 1/6886; G01N 33/57426
USPC 435/6.12; 435/7.23

(57) ABSTRACT

The present application provides methods for obtaining single cells from a sample. Methods for isolating and analyzing molecular features obtained from a single cell are also disclosed herein. For example, individual circulating tumor cells (CTCs) from a sample such as a patient's blood sample can be identified and obtained using methods disclosed herein, and picked for further analysis.

### RELATED APPLICATIONS

The present application claims priority under 35 U.S.C. §119(e) to U.S. Provisional Application Nos. 61/435704, filed Jan. 24, 2011; 61/435724, filed on Jan. 24, 2011; and 61/435721, filed on Jan. 24, 2011. The contents of each of these related applications are herein expressly incorporated by reference in their entirety.

### STATEMENT REGARDING FEDERALLY SPONSORED R&D

This invention, in part, was made with government support under grant U54CA143906 from the National Cancer Institute.

### PARTIES OF JOINT RESEARCH AGREEMENT

This invention, in part, was made under a Research Funding and Option Agreement dated Jun. 25, 2009 with The Scripps Research Institute.

### BACKGROUND

1. Field

The present application relates to the field of cell biology and medicine. More particularly, disclosed herein are methods of obtaining and analyzing single cells from a sample. Also disclosed herein are methods for evaluating the condition of a patient, predicting treatment outcome, and monitoring response to medication by analyzing physical, chemical and/or molecular features obtained from single cells from the patient.

2. Description of the Related Art

Various types of rare cells have been identified in blood and other body fluids. Some of those rare cells can be used to diagnose, monitor, and screen unusual or abnormal conditions, such as pregnancy, infectious diseases and cancer.

Cancer is a difficult disease to treat and manage for several reasons. First, tumor biology changes over the course of the disease. It is fairly common that patients respond well to certain therapies initially, but develop clinical evidence of cancer resistance after being on the therapy for a while. It has been hypothesized that the tumor biology changes due to, for example, genetic instability and pathway shift in response to therapy selection pressure. This necessitates tools for periodic re-assessment of the tumor biology. Second, heterogeneity is a characteristic trait of cancer. As a result, the effectiveness of cancer therapy varies significantly among patients. For a particular cancer treatment, some patients may benefit, but others may suffer severe side effects without much real benefit. Even within the same tumor, tumor cells are often different and their response to chemotherapy may vary.

Circulating tumor cells (CTCs) are cells that have detached from a primary tumor and circulate in the bloodstream. CTCs are thought to be the seed of subsequent growth of additional tumors (metastasis) in different tissues. As such, CTCs can provide a real-time window into the biology of a patient's tumor and facilitate our understanding of the metastatic cascade by studying the evolution of cancer. Detection and characterization of CTCs can also be valuable for stratifying cancer patients and aiding with individualized treatment strategies.

A variety of technologies have been developed for capturing rare cells from biological samples, for example CTCs, from patients. Presently, however, the existing technologies do not allow, for example, capturing individual CTCs for downstream physical, chemical and molecular characterizations. There is a need for methods for obtaining individual CTCs with minimal disruption of the cells and methods for studying single CTCs for determining tumor change over time and heterogeneity of cancer diseases.

### SUMMARY OF THE INVENTION

Some embodiments provided a method for obtaining individual circulating tumor cells (CTCs) in blood, where the method comprises providing a blood sample from a patient; identifying one or more CTCs in the blood sample; and obtaining single CTCs.

In some embodiments, the method comprises lysing non-CTC cells. In some embodiments, the non-CTC cells comprise red blood cells.

In some embodiments, said identifying one or more CTCs comprises an immunochemical analysis. In some embodiments, said identifying one or more CTCs comprises detecting the expression of at least one tumor-specific marker.

In some embodiments, the tumor specific marker is cytokeratin, prostate-specific antigen (PSA), prostate specific membrane antigen (PSMA), mucin-1 (MUC-1), human epidermal growth factor receptor 2 (HER2), AFP (α-fetoprotein), N-cadherin, epithelial cell adhesion molecule (EpCAM), or carcinoembryonic antigen (CEA). In some embodiments, the tumor specific marker is cytokeratin or EpCAM. In some embodiments, the tumor specific marker is an epithelial cell specific marker.

In some embodiments, said identifying one or more CTCs comprises determining the expression of one or more markers that are not expressed in tumor cells.

In some embodiments, said identifying one or more CTCs comprises disposing the sample on a solid support. In some embodiments, the solid support is a non-metallic solid support. In some embodiments, the solid support is a glass slide.

In some embodiments, said obtaining single CTCs comprises separating the CTCs from the solid support. In some embodiments, said separating the CTCs comprises use of a laser capture microdissection (LCM) system or an automated cell picking device. In some embodiments, said separating the CTC comprises removing a single CTC and the portion of the solid support which the single CTC is attached onto from the solid support. In some embodiments, said obtaining the single CTCs comprises aspiration of a single CTC. In some embodiments, the aspiration is based on hydrostatic force. In some embodiments, the aspiration comprises pipetting.

Some embodiments provide a method for assessing cancer progression in a patient suffering from cancer, where the method comprises: providing a circulating tumor cell (CTC) or a substantially pure population of CTCs from the patient; and performing one or more cellular or molecular analyses on the CTCs to determine cancer progression in the patient.

In some embodiments, the substantially pure population of CTCs comprises no more than 20% of non-CTC cells. In some embodiments, the substantially pure population of CTCs comprises no more than 10% of non-CTC cells. In some embodiments, the substantially pure population of CTCs comprises no more than 5% of non-CTC cells.

In some embodiments, the cancer is selected from the group consisting of lung cancer, esophageal cancer, bladder cancer, gastric cancer, colon cancer, skin cancer, papillary thyroid carcinoma, colorectal cancer, breast cancer, lymphoma, pancreatic cancer, prostate cancer, ovarian cancer, pelvic cancer, and testicular cancer.

In some embodiments, said one or more cellular or molecular analysis comprise morphological analysis, genomics analysis, epigenomics analysis, transcriptomics analysis, proteomics analysis, or any combination thereof. In some embodiments, said one or more cellular or molecular analysis comprise determining one or more DNA mutations in the CTCs.

In some embodiments, the DNA mutation comprises an insertion, a deletion, a substitution, a translocation, a gene amplification, or any combination thereof. In some embodiments, the DNA mutation is located in a gene selected from the group consisting of KRAS, BRAF, PTEN, EGFR, ERCC1, RRM1, ELM4, HER2, and ALK. In some embodiments, the DNA mutation is an EML4-ALK fusion or a gene amplification in Her2.

In some embodiments, said one or more cellular or molecular analysis comprise determining protein expression level of a cancer specific gene in the CTCs. In some embodiments, said one or more cellular or molecular analysis comprise determining RNA expression level of a cancer specific gene in the CTCs.

In some embodiments, the cancer specific gene is cytokeratin, prostate-specific antigen (PSA), prostate specific membrane antigen (PSMA), mucin-1 (MUC-1), human epidermal growth factor receptor 2 (HER2), AFP (α-fetoprotein), N-cadherin, epithelial cell adhesion molecule (EpCAM), epidermal growth factor receptor (EGFR), ERCC1, androgen receptor (AR), human equilibrative nucleoside transporter 1 (hENT1), RRM1, or carcinoembryonic antigen (CEA). In some embodiments, the cancer specific gene is an epithelial mesenchymal transition (EMT) marker or a cancer stem cell (CSC) marker. In some embodiments, the EMT maker is selected from the group consisting of N-cadherin, vimentin, B-catenin (nuclear localized), Snail-1, Snail-2 (Slug), Twist, EF1/ZEB1, SIP1/ZEB2, and E47. In some embodiments, the CSC marker is CD133 or CD44.

In some embodiments, said one or more cellular or molecular analysis comprise whole-genome analysis of the CTCs.

Some embodiments provide a method for assessing response of a patient suffering from cancer to a treatment, the method comprises: providing a circulating tumor cell (CTC) or a substantially pure population of CTCs from the patient; and performing one or more cellular or molecular analyses to determine treatment response in the patient.

In some embodiments, the method the substantially pure population of CTCs comprises no more than 20% of non-CTC cells. In some embodiments, the method the substantially pure population of CTCs comprises no more than 5% of non-CTC cells

### BRIEF DESCRIPTION OF THE DRAWINGS

FIG. 1 shows a schematic illustration of a non-limiting embodiment of the CTC-picking methods that is in the scope of the present application.

FIG. 2 is a titration curve resulted from a qPCR assay on a single pancreatic cell PANC1.

FIG. 3 is a gel image showing the amplification result of a qPCR assay on a single pancreatic cell PANC1.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### Definitions

As used herein, the term “rare cells” refers to rare occurring cells in the blood of a human being or other animal subject. For example, the rare cells can be cells that are not normally present in blood, but may be present in blood as a result of an unusual or abnormal condition, such as pregnancy, infectious disease, chronic disease, or injury. Rare cells can also be cells that may be normally present in blood, but are present with a frequency several orders of magnitude less than cells typically present in a normal blood specimen. In some embodiments, the rare cells are more fragile than the other cells that are normally present in blood (e.g., white blood cells and/or red blood cells). Examples of rare cells in blood include, but are not limited to, circulating tumor cells (CTCs), circulating endothelial cells (CECs), fetal cells, stem cells, and any combination thereof. In some embodiments, the rare cell is a CTC. In some embodiments, the rare cell is a fetal cell. In some embodiments, the rare cell is a stem cell.

As used herein, the term “enrichment” refers to the process of substantially increasing the ratio of a target bioentity (e.g., rare cells in blood) to non-target materials in the processed analytical sample compared to the ratio in the original biological sample. In some embodiments, rare cells can be enriched so that the ratio of the rare cells and the non-target material in the blood (e.g., white blood cells) is increased by at least about 10 fold, at least about 100 fold, at least about 500 fold, at least about 1000 fold, at least about 2000 fold, or at least about 5000 fold.

As used herein, the term “substantially pure population of CTCs” refers to a cell population where at least about 60% of the cells are CTCs. In some embodiments, the substantially pure population of CTCs contains no more than about 30%, no more than about 25%, no more than about 20%, no more than about 15%, no more than about 10%, no more than about 5%, no more than about 4%, no more than about 3%, no more than about 2%, no more than about 1%, no more than about 0.5% non-CTCs. In some embodiments, at least about 70%, at least about 75%, at least about 80%, at least about 85%, at least about 90%, at least about 95%, at least about 96%, at least about 97%, at least about 98%, or at least about 99% of the cells in the substantially pure population of CTCs are CTCs.

Disclosed herein are methods for obtaining single cells from a sample. Also disclosed are methods for analyzing physical, chemical and/or molecular features of single cells such as CTCs, and methods for evaluating the condition of a patient, predicting treatment outcome, and/or monitoring response to medication by analyzing physical, chemical and/or molecular features obtained from single cells from the patient.

### Single Cells

As disclosed herein, the single cells can be any desired cells, including rare cells in the sample, such as circulating tumor cells (CTCs). Non-limiting examples of the sample include any biological samples such as blood, lymph, and other body fluids. Various types of rare cells have been identified in body fluids such as blood. Some of those rare cells can be used to diagnose, monitor, and screen unusual or abnormal conditions, such as pregnancy, infectious diseases and cancer.

As a non-limiting example of the rare cells in blood, circulating tumor cells (CTCs) are cells that have detached from a primary tumor and circulate in the bloodstream. CTCs are thought to be the seed of subsequent growth of additional tumors (metastasis) in different tissues. As such, composition of the CTC population, their mechanism of entry into and departure from the bloodstream, metastatic potential of various subsets of CTCs, and the significance of CTCs for patients with early- and late-stage cancers are all important questions to investigate for developing more effective and individualized treatment for cancer patients.

Characterization of CTCs can provide valuable information for stratifying cancer patients and aiding with individualized treatment strategies. For example, the number and/or change in number of detectable CTCs can be used to predict patient outcome and response to therapy. Also, CTCs can be used to identify genetic alterations in tumor cells that impact therapy decisions. In addition, the ability to detect, quantify, or evaluate molecule features of CTCs within a patient's bloodstream can allow genetic manipulations of cell characteristics and/or changing cell behavior while CTCs are en route to the metastatic site and thus altering patient outcome. Further analysis, for example via genomics, epigenomics, transcriptomics, and/or proteomics methods, of CTCs will also help clinicians understand the tumor biology in real-time.

In addition, CTCs can be used to study responses of cancer cells to therapeutic pressure, and discover novel biomarkers and drug targets for cancers.

### Detection and Capture of CTCs

CTCs are fairly rare in blood. The only FDA cleared assay for detecting and isolating CTC at this time is the CellSearch® assay from Veridex. In most patients, the CellSearch® assay finds less than five CTCs per 7.5 ml of blood. A number of technologies have been developed for obtaining CTCs from blood. Most of these technologies use enrichment methods exploit cell surface markers (e.g., EpCAM expression), cell size or cell density.

CellSearch® uses magnetic nanobeads that are coated with anti-EpCAM antibody to capture CTCs in blood. The nanobeads can be first mixed with patient blood. The nanobeads bind to CTCs and are can be pulled out of the blood sample by external magnets. The captured cells are stained with the fluorescently labeled antibodies and dyes listed in Table 1.

TABLE 1
Makers/Labels used in CellSearch ® for obtaining CTCs

| Label | CTC | Leukocyte |
|---|---|---|
| DAPI (nucleus) | Positive | Positive |
| CD45 | Negative | Positive |
| Cytokeratin | Positive | Negative |

The CellSearch® assay results demonstrate the clinical utility of counting CTCs in a patient sample as a prognosis marker. For example, they show with metastatic breast cancer patients, a CTC count of 5 or more per 7.5 ml of blood is predictive of shorter progression free survival and overall survival. Although this utility has been adopted clinically, it provides little knowledge of the tumor biology.

The microfluidic “CTC-chip” technology developed by Toner et al. uses microstructures (posts or herringbone structures) in a microfluidic channel coated with anti-EpCAM antibody and a membrane microfilter device for CTC capture. A blood sample is passed through the microchannels, and CTCs are captured by the microstructures and stained with the same set of fluorescence labels (DAPI, CD45, and cytokeratin). These CTC-chips use a membrane microfilter device for CTC capture, as described in Zheng et al, J. Chromatogr. A., 1162(2):154-161 (2007).

The existing CTC detection and capture technologies described above are disadvantageous for downstream analysis for a number of reasons. For example, these technologies rely on enrichment, e.g., enrichment based on the size difference between tumor cells and white blood cells. As a result of the enrichment step, some true CTCs are lost, while some non-CTCs in the blood sample (e.g., white blood cells) are captured. Also, the cells captured by these technologies are not 100% CTCs. Often times, the CTCs are captured with white blood cells, and as a result, the obtained cell population is a mixture of CTCs and white blood cells. As heterogeneity is a characteristic trait of cancer, studying the CTCs individually will allow a greater understanding the heterogeneity of tumor biology; however, none of the existing technologies allow analysis of individual CTCs. For example, with the CTC-chip technology, after cells are captured by the structures (posts or herringbones) in the microfluidic channels, all captured cells are lysed together to collect nucleic acid of interest for analysis. See, e.g., Stott et al, Proc. Natl. Acad. Sci. USA., 107(43):18392-18397 (2010); Nagrath et al, Nature, 450(7173):1235-1239 (2007). Since the captured cells are in a mixed population of CTCs and non-CTCs (e.g., leukocytes) from the blood sample, no analysis on a single CTC has been possible.

### CTC Assays

Provided herein are methods for identifying and obtaining individual cells, for example CTCs, from a biological sample. Some embodiments provide methods for obtaining individual CTCs in blood, where the methods include providing a blood sample from a patient, identifying one or more CTCs in the blood sample, and obtaining single CTCs from the sample. In some embodiments, the method includes lysing non-CTC cells in the sample, such as red blood cells. In some embodiments, the method includes lysing non-nucleate cells in the sample. In some embodiments, the method does not include lysing non-CTC cells or non-nucleate cells.

A variety of assays can be used herein to identify CTCs in the sample. For example, one non-limiting example of the CTC assay is described in Marrinucci et al., Arch. Pathol. Lab. Med., 133:1468-1471 (2009), in which immunofluorescent staining techniques are used to identify, enumerate, and relocate CTCs from a patient blood sample. In this assay, after lysing red blood cells and centrifugation, the nucleated cell pellet is re-suspended, and the cell solution is dispensed onto microscope glass slides. Cells are then fixed with, for example, formaldehyde, paraformaldehyde, dithio-bis(succinimidyl proprionate), or glutaraldehyde, permeablized with cold methanol, and incubated with a blocking reagent before adding two antibodies that allow differentiation of CTCs and normal blood cells. CTCs are characterized as cytokeratin positive with a nuclear stain such as DAPI or Ethidium Bromide, for example. Cytokeratin expression is used widely in diagnostic tumor pathology to identify a neoplasm as epithelial in nature. The white blood cell specific antibody, anti-CD45, is used to differentiate white blood cells from CTCs (which are CD45 negative). Additional non-limiting examples of methods of detecting cancer cells useful in the embodiments disclosed herein are described in Kraeft et al., Clin. Cancer Res. 6: 434 (2000) and Krivacic et al., Proc. Natl. Acad. Sci. USA 101:10501-10504 (2004).

In some embodiments, CTCs can be identified via immunochemical analysis. For example, CTCs can be identified by detecting the expression of one or more tumor-specific markers. In some embodiments, the expression of a tumor-specific marker is determined by detecting the presence or absence of the tumor-specific marker on cell surface of the cells in a sample (e.g., CTCs and non-CTC cells). Non-limiting examples of tumor specific markers useful in the embodiments disclosed herein include cytokeratin, prostate-specific antigen (PSA), prostate specific membrane antigen (PSMA), mucin-1 (MUC-1), human epidermal growth factor receptor 2 (HER2), AFP (α-fetoprotein), N-cadherin, epithelial cell adhesion molecule (EpCAM), or carcinoembryonic antigen (CEA). In some embodiments, the tumor specific marker is an epithelial cell specific marker. In some embodiments, the tumor specific marker is cytokeratin or EpCAM. One of ordinary skill in the art will appreciate that any suitable methods, such as immunochemical methods, can be used to detect the presence or absence of the expression of the marker in or on the surface of the cells. For example, an antibody capable of specifically recognizing the tumor specific markers can be used, or ligands capable of specifically binding to the tumor specific cell surface molecules can be used (e.g., epidermal growth factor).

In some embodiments, the sample is treated with an agent that labels nuclei. Non-limiting examples of such agent include 4′,6-diamidino-2-phenylindole (DAPI) and Ethidium Bromide. In some embodiments, CTCs can be differentiated from non-CTCs, and thus be identified, by detecting one or more markers that are not expressed in CTCs, but expressed in one or more types of non-CTCs (e.g., leukocytes). For example, identification of CTCs can include determining whether a cell is CD45 positive or negative.

In some embodiments, the sample is disposed on a solid support for identifying and/or obtaining CTCs. Examples of the solid support include, but are not limited to, microfluidic chip, a silicon chip, a microscope slide, a glass slide, a glass microscope slide, a microplate well, a polymeric membrane, a derivatized plastic film. In some embodiments, the solid support is non-metallic. In some embodiments, the solid support is substantially transparent. In some embodiments, the solid support is a glass microscope slide. In some embodiments, the CTCs are identified using a microscope. In some embodiments, identification of the CTCs includes a microscopic scan of the sample.

For example, the sample can be disposed on a glass substrate to allow the cells in the sample to adhere to the glass substrate through electrostatic interactions. In some embodiments, the cells, for example CTCs, can be removed from the slides with mechanical force. In some embodiments, the glass substrates (e.g., glass slides) can be modified with different coating. For example, certain reversible chemical bonds can be created on the glass slides, so that the cells can adhere to the glass slides and go through the detection assay (e.g., immunochemical assay) on the glass slides. Releasing reagent can be applied to reverse the chemical bond to release the cells from the glass slides and allow picking of individual cells. In some embodiments, the cell picking process is automated.

In some embodiments, the density of cells on the slides can be maximized to reduce the number of slides for a given sample (e.g., a blood sample). In some embodiments, the loading density of cells can be reduced to allow automated cell picking.

In some embodiments, the method allows obtaining rare cells, for example individual rare cells, without significant disruption of the cells. Therefore, these methods allow preservation of cytological details of the cells and detailed downstream analysis of the cells.

In some embodiments, the cells in the sample are disposed on the solid support as a monolayer. In some embodiments, the sample is contacted with a fixative to fix the cells on to a support. Non-limiting of the fixative include reversible cross-linking fixatives, formaldehyde, formalin, paraformaldehyde, dithio-bis(succinimidyl proprionate) (DSP), and glutaraldehyde.

### Cell Picking

Also disclosed herein are methods for obtaining individual cells, such as CTCs from a sample. A variety of cell picking techniques can be used herein. In some embodiments, after being identified, individual CTCs can be separated from non-CTCs in the sample on the solid support.

For example, a microinjection system can be mounted on a micromanipulation system for cell picking. In some embodiments, the micromanipulation system can be mounted on a microscope stage for cell picking. For example, Eppendorf's microinjection system CellTram Vario can be mounted on a non-limiting example of the micromanipulation system, Eppendorf TransferMan NK2. The blood sample can, for example, be disposed on a glass slide. Before cell picking starts, all CTCs on the glass slide can be relocated on a fluorescence microscope. After removing nailpolish from the glass slide, the glass slide can be soaked in PBS buffer to let the coverslip float away. The glass slide can then be soaked in methanol to dissolve the glycerol based mounting media. To perform CTC picking, the glass slide can be covered with BSA solution to help loosen the adhesion of CTCs on the microscope glass slide and significantly reduce the stiction of CTCs to glass capillaries used for picking.

Laser capture microdissection (LCM) is another nonlimiting example of a cell picking method. LCM is also known as microdissection, laser microdissection (LMD), or laser-assisted microdissection (LAM). In LCM, a laser can be coupled to a microscope and focused onto a sample on a slide. The components and use of the LCM system are well known in the art, for example, described in US Publication No. 20100157284. In this method, the laser can be directed to follow a trajectory predefined by a user to cut out a selected subset of a sample on a slide. In some embodiments, the selected subset can be separated from the remainder of the slide sample using, for example, contacting the selected subset with an adhesive, melting a plastic membrane onto the surface of the selected subset and tearing out the selected subset, precise transport by Laser Pressure Catapult or laser-induced forward transfer, or transport by simple gravity. As a nonlimiting example, the Applied Biosystems Arcturus LCM Instrument can be used.

In some embodiments, an automated cell picking device can be used. In some embodiments, the cell picking device comprises an automated imaging apparatus and a cell-picking apparatus. The cell-picking apparatus can be configured to pick a cell identified by the imaging apparatus. In some embodiments, the cell picking apparatus can be understood as a robot for cell picking having an integrated imaging camera. A cell picking head is provided that comprises a hollow pin for aspirating a single cell such as a mammalian CTC cell, allowing a cell to be picked from a microscope slide. The cell picking head can suspended over the slide by way of a head positioning system made up of x-, y- and z-linear positioners operable to move the cell picking head over the slide. All movements can be controlled by the controller.

Other methods of separating a subset from a sample on a solid support (e.g., a slide) are also contemplated or can be obvious to one of ordinary skill in the art. In some embodiments, one or more CTCs can be separated from non-CTCs by separating a portion of the solid support that contains no non-CTCs from the remainder of the solid support. For example, a portion of a slide containing a single CTC can be cut and separated from the remainder of the slide. In some embodiment, the solid support is a glass slide.

The CTCs can also be separated from non-CTCs in the sample by aspiration of a single CTC. For example, pipetting can be used to collect a cell from the face of the solid support (e.g., a microscope slide). In some embodiments, a hydrostatic reaction or force facilitates separation of a cell from a slide. In some embodiments, the aspiration comprises pipetting or use of a microcapillary, for example a glass microcapillary. In some embodiments, a micromanipulator or a pipette is used to remove CTCs from the solid support one CTC at a time. Another non-limiting example of the cell picking methods includes coating a glass capillary with silicone. In this method, individual CTCs or multiple CTCs can be aspirated into a glass capillary.

The methods disclosed herein are advantageous in several aspects. For example, they allow isolation of single CTCs as well as substantially pure CTC populations from a sample and permit the placement of the cells in any format that is compatible with downstream analysis. For example, the CTCs can be credentialed with immunofluorescence techniques and pathological review, and the isolated CTCs are not contaminated with any other white blood cells. Further, the methods allow studying of CTCs individually. For example, a single CTC from a patient sample can be retrieved and analyzed with molecular technologies such as PCR, sequencing, and others. Moreover, the ability to obtain single CTCs and a cell population with high purity level of CTCs can, for example, significantly decrease false negative in cancer diagnosis, prognosis, and facilitate studies in therapy response.

As described herein, in some embodiments, a minimally invasive CTC assay is used to capture and identify CTCs. In some embodiments, cell morphology of the CTC is minimally altered. In some embodiments, a single CTC cell is isolated. In some embodiments, the CTC obtained using the methods described herein is intact. It will be appreciated by one of ordinary skill in the art that it is advantageous to obtain intact CTCs or CTCs with minimally altered cell morphology to allow high-quality images with preserved cellular details for pathological review. In some embodiments, automated fluorescence imaging systems are used to determine the location of the CTC. For example, automated fluorescence imaging system can be used in some embodiments to determine and record the exact locations (X and Y coordinates) of the identified CTCs on the solid support (e.g., a microscope slide).

Some embodiments provided herein include a step of enriching the rare cells, such as CTCs, in the sample. In some embodiments, the enrichment step occurs before the step of obtaining the individual rare cells. For example, before picking for CTCs, the sample can be enriched for CTCs. A variety of methods are known in the art to enrich predetermined cells in a sample. Such methods have been used to enrich fetal cells from a sample of maternal peripheral blood and tumor cells from bodily fluid. For example, cell sorting by FACS technology has been applied to enumerate and collect rare cells in biological samples. Several immunochemical methods, including immunocapturing methods, have also been developed for the enrichment of cells from fluid specimens using solid phase absorption. U.S. Patent Publication No. 20100285581 describes methods for enriching cells of interest with high purity based on solid phase isolation (which is hereby incorporated by reference in its entirety). Skilled artisan will appreciate that any suitable methods known in the art can be used to enrich rare cells in the methods and kits disclosed herein.

The CTC cell population obtained using the method disclosed herein, in general, contains low contamination of non-CTC cells. In some embodiments, the CTC cell population obtained using the methods disclosed herein contains no more than about 20%, no more than about 15%, no more than about 10%, no more than about 5%, no more than about 4%, no more than about 3%, no more than about 2%, no more than about 1%, or no more than about 0.5% non-CTC cells.

### Single Cell Genomics

Nucleic acid analysis can be done at the single cell level. For example, microfluidics-based technology for single cell mRNA isolation and analysis has been developed. The nCounter^{TM} gene expression system for direct multiplexed measurement of gene expression with color-coded probe pairs without amplification that was developed by NanoString Technologies also has the potential for single cell transcriptomics.

Great progress has been made in next generation sequencing technologies. For example, Pacific Biosciences have developed a technology for single-molecule and real-time DNA sequencing by a single DNA polymerase; Helicos Biosciences have developed a high-throughput, amplification-free method for transcriptome quantification; and Oxford Nanopore Technologies have developed a single-molecule sequencing technology using nanopores.

While progress has been made in single cell analysis, it remains a significant challenge to select, isolate, and manipulate single cells from biological samples. As such, there is still a need to develop new technologies to enable direct single cell omics applications.

As described above, the methods disclosed herein allow obtaining single CTCs and substantially pure population of CTCs from biological samples, such as blood. Using methods described herein, single CTCs and the CTC cell population can be identified and obtained to allow downstream analysis, for example, physical, chemical (e.g., biochemical), and/or molecular analysis. Various techniques can be used to conduct these studies to analyze physical, chemical and/or molecular features (e.g., DNA, RNA, microRNA, DNA methylation, and protein) of the CTCs. Examples of the analysis include, but are not limited to cytomorphological analysis, genomics analysis, proteomics analysis, transcriptomics analysis, epigenomics analysis, and any combinations thereof. In some embodiments, the analysis is performed on a single CTC. In some embodiments, the analysis is performed on a substantially pure population of CTCs.

Some embodiments provide a method for assessing cancer progression in a patient suffering from cancer, where the method include providing a circulating tumor cell (CTC) or a substantially pure population of CTCs from the patient and performing one or more cellular or molecular analyses on the CTCs to determine cancer progression in the patient. The amount of non-CTC cells in the substantially pure population of CTCs can vary. For example, the substantially pure population of CTCs can include no more than 20% of non-CTC cells, no more than 10% of non-CTC cells, or no more than 5% non-CTC cells. As used herein, non-limiting examples of cellular analysis include counting the number of the CTCs, cytomorphological analysis of the CTCs, and other techniques available for studying cellular details of cells.

The types of cancer that the CTCs can be used for diagnosis and prognosis are not particularly limited. The cancer can be, for example, lung cancer, esophageal cancer, bladder cancer, gastric cancer, colon cancer, skin cancer, papillary thyroid carcinoma, colorectal cancer, breast cancer, lymphoma, pancreatic cancer, prostate cancer, ovarian cancer, pelvic cancer, and testicular cancer.

In some embodiments, one or more molecular features of the CTCs are analyzed. Examples of the molecular features include, but are not limited to, nucleic acid composition, protein composition, DNA methylation profile, protein glycosylation, and phosphorylation pattern. In some embodiments, nucleic acids (e.g., DNAs and RNAs) of the CTCs are isolated and analyzed. In some embodiments, whole genome amplification is performed before the molecular analysis. In some embodiments, the DNA sequence in cancer mutation hotspots in the CTCs is determined. Non-limiting examples of cancer mutation hotspots include mutation hotspots in genes such as Ras, p53, Braf, Pten, Egfr, Ercc1, Rrm1, Elm4, Alk, and Her2 gene. In some embodiments, the CTCs are analyzed for the presence or absence of gene amplification or translocation. For example, the CTCs can be analyzed to determine the presence or absence of Elm4-Alk translocation.

The results obtained from the physical, chemical, and molecular analysis of CTCs can provide valuable information for various applications including, but not limited to, evaluating condition of the cancer patient, assessing or predicting cancer progression, assessing or predicting treatment response of the cancer patient, cancer prognosis, screening targets for cancer drugs, predicting treatment outcome, discovering novel biomarkers, and understanding response of cancer cell to therapeutic pressure.

Examples of methods that can be used for downstream analyses to characterize and/or analyze the cells include, but are not limited to, biochemical analysis; immunochemical analysis; image analysis; cytomorphological analysis; molecule analysis such as PCR, sequencing, determination of DNA methylation; proteomics analysis such as determination of protein glycosylation and/or phosphorylation pattern; genomics analysis; epigenomics analysis; transcriptomics analysis; and any combination thereof. In some embodiments, molecular features of the CTCs are analyzed by image analysis, PCR (including the standard and all variants of PCR), microarray (including, but not limited to DNA microarray, MMchips for microRNA, protein microarray, cellular microarray, antibody microarray, and carbohydrate array), sequencing, biomarker detection, or methods for determining DNA methylation or protein glycosylation pattern. Some non-limiting examples of these analyses are shown in Table 2. In some embodiments, the CTCs are analyzed by quantitative PCR (qPCR) (e.g., real-time quantitative PCR) and RT-PCR. In some embodiments, nucleic acid composition, protein composition, DNA methylation profile, and/or protein glycosylation and/or phosphorylation pattern of a single CTC can be analyzed.

TABLE 2
Examples of Molecular Analysis

| | DNA | RNA | Protein |
|---|---|---|---|
| PCR | Mutations in | Gene expression of | |
| | target genes | target genes | |
| Microarray | Mutations in | Gene expression of | |
| | target genes | target genes | |
| Target | Mutations in | | |
| Sequencing | target genes | | |
| Next-Gen | Mutations in | Gene expression of | |
| Sequencing | target genes or | target genes or whole | |
| | whole genome | transcriptome | |
| | | analysis | |
| Mass | | | Protein detection |
| Spectrometry | | | and |
| | | | quantification |

In some embodiments, the single cells are from a patient suffering from cancer. In some embodiments, the single cells are from a subject suspected of cancer. In some embodiment, the cancer patient is receiving or has been treated with cancer treatment(s). In some embodiments, the CTCs are obtained from a blood sample. In some embodiments, the CTCs are from body fluid.

In some embodiments, the methods allow obtaining individual CTCs without significant disruption of the cells. Therefore, these methods allow preservation of cytologic details of the cells and detailed downstream analysis of the CTCs. Any suitable methods known in the art can be used to determine the structural integrity of the rare cells. Non-limiting examples of such methods include immunocytochemical procedures, fluorescence in situ hybridization (FISH), flow cytometry, image cytometry, and any combinations thereof.

Cellular heterogeneity within isogenic cell population is a widespread event in cell biology. Analyzing cell ensembles individually will lead to a more accurate representation of cell-to-cell variations. To that end, a lot of focus has been on developing technologies for single cell genomics, transcriptomics, epigenomics, and proteomics.

Similar to the cells in a primary tumor, CTCs from a patient blood sample can also be heterogeneous. Understanding the heterogeneity of CTCs will allow categorization of the CTCs into subpopulations based on one or a set of biomarkers. For example, while not wishing to be bound to any particular theory, it is hypothesized that once tumor cells get into blood circulation, some of them go through an epithelial-mesenchymal transition (EMT). Analysis of the expressions of a set of epithelial and mesenchymal markers in this subpopulation of CTCs will lead to a deeper understanding of the role of EMT in cancer metastasis.

The methods disclosed herein allow studying the distribution of the markers of interest (for example, mutation, gene expression, protein, DNA methylation, regulatory RNA (e.g., miRNA and siRNA), and etc.) among the CTCs.

Understanding the heterogeneity of CTCs will also allow development of scoring algorithms to determine the status of biomarkers. For example, in order to find out whether certain patients are positive for KRAS mutations, one can first determine how many positive CTCs from one patient have to be detected before the patients are considered positive by detecting and quantifying CTCs in known cancer patients.

Understanding the relevant DNA, RNA, and protein markers in the CTCs from cancer patients and correlating them with patients' clinical information is also of importance in cancer biomarker discovery, cancer diagnosis, prognosis, and therapy monitoring.

Genomics, epigenomics, transcriptomics, and proteomics analysis of single CTCs will provide a real-time window into the biology of a tumor and facilitate an understanding of tumor biology in real-time.

For example, the condition of a cancer patient can be evaluated by analyzing sequence information obtained from a CTC. The sequence information can include insertion/deletion/mutation of the genomic sequence, methylation pattern of the DNA, and epigenetic characteristic of the DNA. In some embodiments, the condition of a cancer patient can be evaluated by analyzing biochemistry information obtained from a CTC. The biochemistry information can include information regarding protein glycosylation, protein phosphorylation and other post-translational modification on proteins.

In some embodiments, one or more gene mutations in the CTCs are determined. The types of gene mutation are not particularly limited. Non-limiting examples of gene mutation include insertions, deletions, substitutions, translocations, gene amplifications, and any combinations thereof. In some embodiments, the gene mutation is located in KRAS, BRAF, PTEN, EGFR, ERCC1, RRM1, ELM4, HER2, or ALK gene. In some embodiments, the DNA mutation is an EML4-ALK fusion or a gene amplification in Her2. In some embodiments, whole-genome analysis of the CTCs is performed.

In some embodiments, protein expression level of a cancer specific gene of the CTCs is determined In some embodiments, RNA expression level of a cancer specific gene of the CTCs is determined. Examples of cancer specific gene include, but are not limited to, cytokeratin, prostate-specific antigen (PSA), prostate specific membrane antigen (PSMA), mucin-1 (MUC-1), human epidermal growth factor receptor 2 (HER2), AFP (α-fetoprotein), N-cadherin, epithelial cell adhesion molecule (EpCAM), epidermal growth factor receptor (EGFR), ERCC1, androgen receptor (AR), human equilibrative nucleoside transporter 1 (hENT1), RRM1, and carcinoembryonic antigen (CEA). Other non-limiting examples of the cancer specific gene include epithelial mesenchymal transition (EMT) markers are cancer stem cell (CSC) markers. Non-limiting examples of EMT markers include N-cadherin, vimentin, B-catenin (nuclear localized), Snail-1, Snail-2 (Slug), Twist, EF1/ZEB1, SIP1/ZEB2, and E47. Examples of CSC markers include, but are not limited to, CD133 and CD44.

The embodiments disclosed herein also include methods for assessing or predict response of a patient suffering from cancer to a treatment, where the methods include providing a circulating tumor cell (CTC) or a substantially pure population of CTCs from the patient and performing one or more cellular or molecular analyses on the CTCs to determine treatment response in the patient. For example, expression levels of HER2 protein was found to correlate significantly with patients' response to anti-cancer drug lapatinib. Single CTCs obtained from a cancer patient using the methods disclosed herein can be analyzed for HER2 protein expression, and the HER2 protein expression level can be used to predict or assess the patient's response to lapatinib treatment and thus can be used in the development of an appropriate treatment regimen. As another example, the presence of cancer stem cell markers such as ALDH, CD44, CD133, and CD 166 correlates with poor prognosis for colorectal cancer patients. However, certain therapies, i.e., dasatinib and curcumin combination therapy, has been shown to significantly reduce the number of cancer stem cells. Accordingly, the isolation and analysis of CTCs for cancer stem cell markers can be used to determine whether it is appropriate to treat a patient with certain chemotherapeutics. As such, methods disclosed herein for isolating single CTCs can be used to develop targeted therapies for cancer patients.

As another example, molecular features (e.g., sequence and biochemistry information) obtained from the CTCs can be used to evaluate the patient's response to a cancer treatment, patient prognosis, patient diagnosis, or remission state of a patient.

### EXAMPLES

Additional embodiments are disclosed in further detail in the following examples, which are not in any way intended to limit the scope of the claims.

### Example 1

### Identification of CTCs in a Blood Sample

Peripheral blood is collected from primary lung cancer patients in a cell-free DNA blood collection tube (Streck, Omaha, Nebr.). A white blood cell count is taken from the blood sample using a hemocytometer, and the cellular concentration of the sample is titrated so that it is about 3 million cells per slide when the titrated sample is disposed on a glass slide. After lysing red blood cells using ammonium chloride, the nucleated cells are distributed in a monolayer onto the glass slide. After paraformaldehyde fixation and methanol permeabilization, cells are incubated with anti-Cytokeratin cocktail and anti-CD45 antibodies followed by Alexa 555-conjugated secondary antibody and DAPI as a nuclear stain.

The glass slide is imaged (custom high speed scanning microscope, Epic Sciences at 10×) and “candidate” CTCs are identified as being Cytokeratin positive (CK+), CD45 negative (CD45−) with an intact nucleus using proprietary computer algorithms (Epic Sciences). Each CTC candidate is subsequently evaluated by direct microscopic review of captured images and based on cell morphology and immunophenotype is either confirmed or rejected as being a CTC by two independent reviewers.

### Example 2

### Capture of Individual CTCs in a Blood Sample

CTCs are identified in a blood sample according to the general procedure described in Example 1. The CTCs on the glass slide are relocated on a fluorescence microscope. The glass slide is soaked in PBS buffer for 30 minutes to let the coverslip float off, and then soaked in methanol for about 1 hour to dissolve the glycerol-based mounting media. To perform CTC picking, the slide is covered with BSA solution which can help loosen the adhesion of CTCs on the glass slide and significantly reduce the stiction of CTCs to glass capillaries used for picking. A micromanipulator mounted on the microscope stage is used to pick CTCs from the slide one CTC at a time. The isolated CTC is put into a tube, either separately or with other isolated CTCs, for downstream analysis.

### Example 3

### Capture of Single CTCs in a Blood Sample

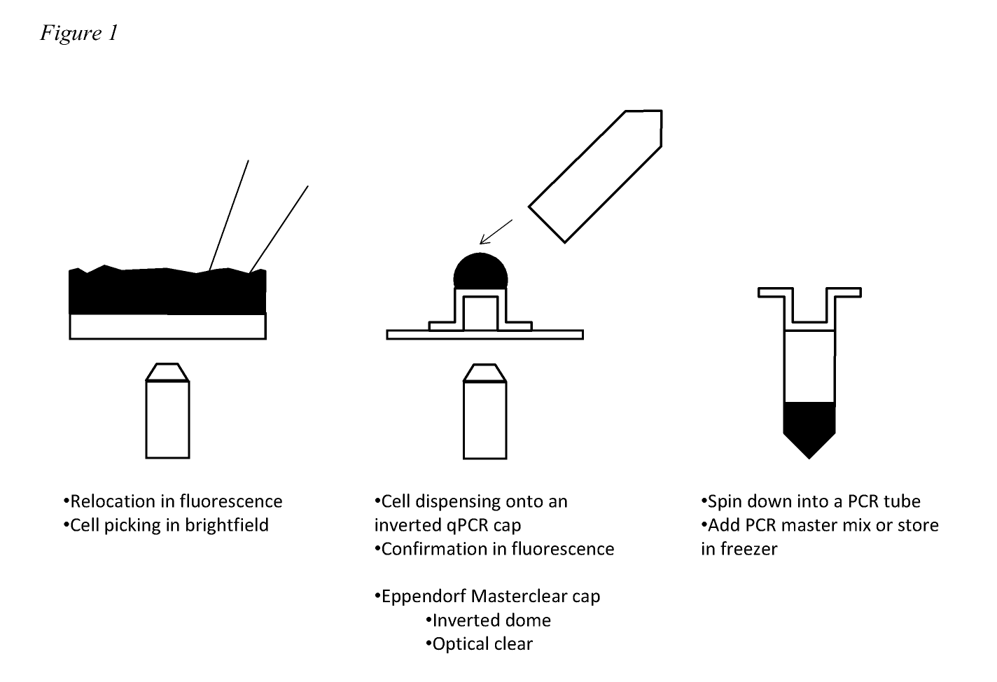

An exemplary embodiment of the method for capturing single CTCs is illustrated in FIG. 1. In this example, transparent qPCR tube cap that allows the detection of fluorescence detection through the cap for real-time PCR is laid upside down on top of glass slide. A small (for example, 1 to 5 μl) droplet of PBS buffer is put into the cap and the aspirated CTCs is dispensed into the PBS droplet. Then, fluorescence detection is performed to allow detection and confirmation of the number of CTCs and the purity of CTCs in the droplet. Finally, the cap is closed with a PCR tube. With a quick spin, the droplet will be at the bottom of the PCR tube.

### Example 4

### Capture and DNA Analysis of Single Pancreatic Cancer Cells in a Blood Sample

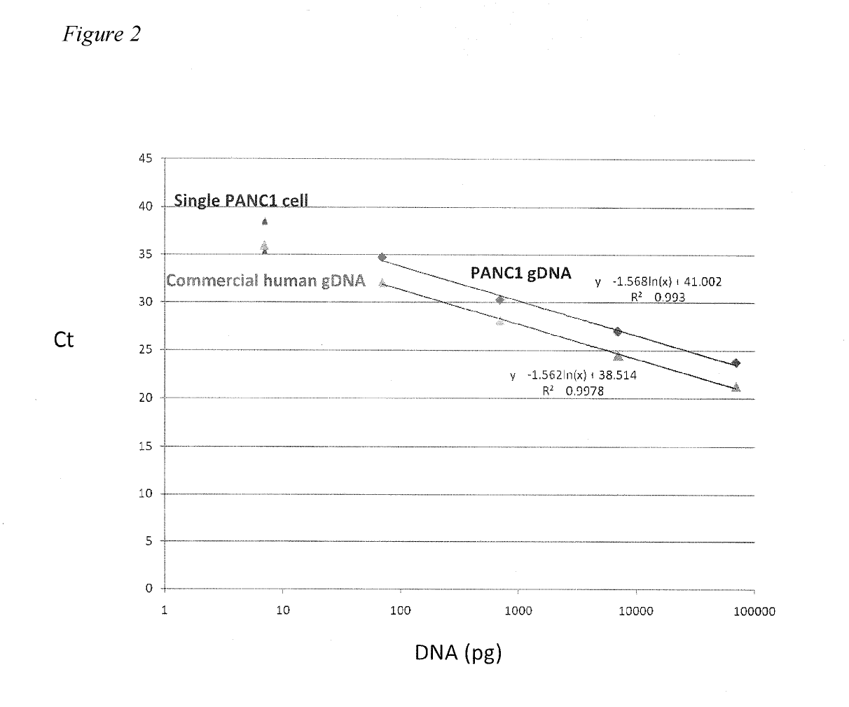

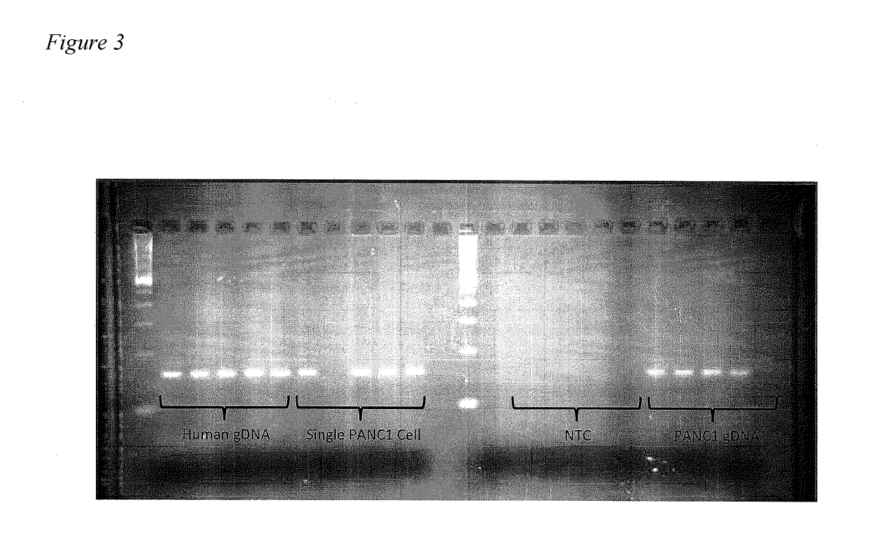

Human pancreatic carcinoma cell line PANC1 cells were spiked into healthy donor blood sample. The sample was processed with the general procedure described in Example 1 and PANC1 cell line cells were identified on the glass slides. A single PANC1 cell was retrieved from the glass slides and put into a 3 ul PBS buffer in a PCR tube. PBS buffer containing no template was used as negative control. Commercially available human genomic DNAs in the amount of 7 pg, 70 pg, 700 pg, 7 ng, and 70 ng were used as positive control. Genomic DNA extracted from PANC1 cell line cells in the similar amounts was used as another positive control. SYBR green based qPCR assay targeting a house-keeping gene was run with 5 replicates of single PANC1 cell and all the controls. The titration curves are shown in FIG. 2, and gel images are shown in FIG. 3. From the slope of the titration curves for two positive controls in FIG. 2, the PCR efficiency was found to be 90%. DNA from four out of the five single PANC1 cells was successfully amplified and the Ct values of the housekeeping gene were similar to the one with equivalent amount of human genomic DNA. Gel images in FIG. 3 confirmed that the amplicon length from single PANC1 cell was similar to the ones from positive controls.

The data demonstrates that single CTCs can be captured, identified, and isolated from the patient blood sample, and a specific DNA target in a single CTC can be amplified and detected with PCR.

### Example 5

### Scanning of CTC Cells on a Microscope Slide

A glass slide on which a blood sample is disposed onto is automatically scanned using a Rare Event Imaging System (Georgia Instruments Inc., Roswell, Ga.). Images are taken by an integrating, cooled CCD detector and processed in a 60-MHz Pentium imaging workstation. In the first step, the slide is automatically scanned for the detection of positive events (e.g., cytokeratin+cells) using the rhodamine filter set. The identification of positive events is based on fluorescence intensity and area. The (X,Y) coordinates of each positive event are stored into computer memory, and the image is archived. In the second step, the slide is scanned for the total number of DAPI-labeled nuclei per slide, representing the total cell count. At the end of the two scans, the number of positive events and the total cell count are given, and a gallery of images containing all positive events is displayed. The user can review the images and recall any of the events for further examination using the stored coordinates attached to each image. The field of interest can then be visualized using higher magnification and additional filter sets (e.g., fluorescein, or UV filter). Images of different fluorescent colors are electronically overlaid for positive confirmation of the event and for phenotypic evaluation (multiple labeling).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for obtaining individual circulating tumor cells (CTCs) in blood, comprising:
providing a blood sample from a patient;
identifying one or more CTCs in the blood sample; and
obtaining single CTCs.

2. The method of claim 1, wherein the method comprises lysing non-CTC cells.

3. The method of claim 2, wherein the non-CTC cells comprises red blood cells.

4. The method of claim 1, wherein said identifying one or more CTCs comprises an immunochemical analysis.

5. The method of claim 1, wherein identifying one or more CTCs comprises detecting the expression of at least one tumor-specific marker.

6. The method of claim 5, wherein the tumor specific marker is cytokeratin, prostate-specific antigen (PSA), prostate specific membrane antigen (PSMA), mucin- 1 (MUC-1), human epidermal growth factor receptor 2 (HER2), AFP (α-fetoprotein), N-cadherin, epithelial cell adhesion molecule (EpCAM), or carcinoembryonic antigen (CEA).

7. The method of claim 5, wherein the tumor specific marker is cytokeratin or EpCAM.

8. The method of claim 5, wherein the tumor specific marker is an epithelial cell specific marker.

9. The method of claim 5, wherein said identifying one or more CTCs comprises determining the expression of one or more markers that are not expressed in tumor cells.

10. The method of claim 1, wherein said identifying one or more CTCs comprises disposing the sample on a solid support.

11. The method of claim 10, wherein the solid support is a non-metallic solid support.

12. The method of claim 10, wherein the solid support is a glass slide.

13. The method of claim 10, wherein said obtaining single CTCs comprises separating the CTCs from the solid support.

14. The method of claim 13, wherein said separating the CTCs comprises use of a laser capture microdissection (LCM) system or an automated cell picking device.

15. The method of claim 13, wherein said separating the CTC comprises removing a single CTC and the portion of the solid support which the single CTC is attached onto from the solid support.

16. The method of claim 1, wherein said obtaining the single CTCs comprises aspiration of a single CTC.

17. The method of claim 16, wherein the aspiration is based on hydrostatic force.

18. The method of claim 16, wherein the aspiration comprises pipetting.

19. A method for assessing cancer progression in a patient suffering from cancer, comprising:
providing a circulating tumor cell (CTC) or a substantially pure population of CTCs from the patient; and
performing one or more cellular or molecular analyses on the CTCs to determine cancer progression in the patient.

20. The method of claim 19, wherein the substantially pure population of CTCs comprises no more than 20% of non-CTC cells.

21. The method of claim 19, wherein the substantially pure population of CTCs comprises no more than 10% of non-CTC cells.

22. The method of claim 19, wherein the substantially pure population of CTCs comprises no more than 5% of non-CTC cells.

23. The method of claim 19, wherein the cancer is selected from the group consisting of lung cancer, esophageal cancer, bladder cancer, gastric cancer, colon cancer, skin cancer, papillary thyroid carcinoma, colorectal cancer, breast cancer, lymphoma, pancreatic cancer, prostate cancer, ovarian cancer, pelvic cancer, and testicular cancer.

24. The method of claim 19, wherein said one or more cellular or molecular analysis comprise morphological analysis, genomics analysis, epigenomics analysis, transcriptomics analysis, proteomics analysis, or any combination thereof.

25. The method of claim 19, wherein said one or more cellular or molecular analysis comprise determining one or more DNA mutations in the CTCs.

26. The method of claim 25, wherein the DNA mutation comprises an insertion, a deletion, a substitution, a translocation, a gene amplification, or any combination thereof.

27. The method of claim 25, wherein the DNA mutation is located in a gene selected from the group consisting of KRAS, BRAF, PTEN, EGFR, ERCC1, RRM1, ELM4, HER2, and ALK.

28. The method of claim 25, wherein the DNA mutation is an EML4-ALK fusion or a gene amplification in Her2.

29. The method of claim 23, wherein said one or more cellular or molecular analysis comprise determining protein expression level of a cancer specific gene in the CTCs.

30. The method of claim 23, wherein said one or more cellular or molecular analysis comprise determining RNA expression level of a cancer specific gene in the CTCs.

31. The method of claim 29, wherein the cancer specific gene is cytokeratin, prostate-specific antigen (PSA), prostate specific membrane antigen (PSMA), mucin-1 (MUC-1), human epidermal growth factor receptor 2 (HER2), AFP (α-fetoprotein), N-cadherin, epithelial cell adhesion molecule (EpCAM), epidermal growth factor receptor (EGFR), ERCC1, androgen receptor (AR), human equilibrative nucleoside transporter 1 (hENT1), RRM1, or carcinoembryonic antigen (CEA).

32. The method of claim 29, wherein the cancer specific gene is an epithelial mesenchymal transition (EMT) marker or a cancer stem cell (CSC) marker.

33. The method of claim 32, wherein the EMT maker is selected from the group consisting of N-cadherin, vimentin, B-catenin (nuclear localized), Snail- 1, Snail-2 (Slug), Twist, EF1/ZEB1, SIP1/ZEB2, and E47.

34. The method of claim 32, wherein the CSC marker is CD133 or CD44.

35. The method of claim 19, wherein said one or more cellular or molecular analysis comprise whole-genome analysis of the CTCs.

36. A method for assessing response of a patient suffering from cancer to a treatment, comprising:
providing a circulating tumor cell (CTC) or a substantially pure population of CTCs from the patient; and
performing one or more cellular or molecular analyses to determine treatment response in the patient.

37. The method of claim 36, wherein the method the substantially pure population of CTCs comprises no more than 20% of non-CTC cells.

38. The method of claim 36, wherein the method the substantially pure population of CTCs comprises no more than 5% of non-CTC cells.
